(19) United States
(12) Patent Application Publication
(10) Pub. No.: US 2008/0066752 A1
(43) Pub. Date: Mar. 20, 2008

(54) Method and system for circulatory delay compensation in closed-loop control of a medical device

(75) Inventors: Clark R. Baker, Newman, CA (US); Joseph Douglas Vandine, Newark, CA (US)

Correspondence Address: Covidien;IP Counsel - Respiratory & Monitoring Solutions, 60 Middletown Avenue, North Haven, CT, 06473, US

(73) Assignee: Nellcor Puritan Bennett Inc.

(21) Appl. No.: 11/524,182

(22) Filed: Sep. 20, 2006

(51) Int. Cl. A61M 16/00

(52) U.S. Cl. 128/204.23; 128/204.21

(57) ABSTRACT

Embodiments of the present invention relate to a system and method for automatically controlling a physiologic parameter of a patient. Specifically, embodiments of the present invention include delivering a gas mixture to the patient and monitoring at least one physiologic parameter of the patient, detecting whether a physiologic delay exists between delivering the gas mixture and detection of a corresponding response to delivering the gas mixture, and automatically controlling the delivery of the gas mixture based on whether the delay is detected and based on a value of the physiologic parameter.

### BACKGROUND OF THE INVENTION

1. Field of the Invention

The present invention relates generally to a method and system for closed-loop control of a medical device based on monitored physiologic parameters. Specifically, embodiments of the present invention relate to adjusting device control features to account for delays (e.g., circulatory delay) in detecting the impact of control manipulation on the monitored physiologic parameters (e.g., blood oxygen saturation).

2. Description of the Related Art

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

Certain patient physiologic conditions may be achieved or maintained using medical devices designed for such purposes. For example, mechanical ventilators may be utilized to maintain a level of oxygenation in a patient's arterial blood or to achieve a desired arterial pH level. These medical devices may be manually or automatically operated and adjusted based on a comparison of measured and desired physiologic parameter values. For example, upon sensing a blood oxygenation level below a desired value in a patient, an operator could manually adjust a ventilator to provide additional oxygen to the patient. In another example, based on detection of blood oxygenation below a desired level in a patient, a device may utilize closed-loop control to adjust itself such that additional oxygen is provided to the patient.

Automated control of patient physiologic parameters may be achieved using various types of control devices and/or algorithms to operate medical devices. For example, computer-based controllers, such as proportional (P) controllers, proportional-integral (PI) controllers, proportional-derivative (PD) controllers, or proportional-integral-derivative (PID) controllers may be utilized to control the output of a medical device based on a measured physiologic parameter value. However, while use of automated controllers to operate medical devices may be more efficient and accurate than manual operation, issues may arise due to delays in the control loop that impact controller response times.

### BRIEF DESCRIPTION OF THE DRAWINGS

Advantages of the invention may become apparent upon reading the following detailed description and upon reference to the drawings in which:

FIG. 1 is a block diagram of a ventilation system that induces, maintains, or controls blood oxygen saturation in a patient while accounting for physiologic delay in accordance with an exemplary embodiment of the present invention; and

FIG. 2 is a block diagram of a method illustrating an exemplary embodiment of the present invention.

### DETAILED DESCRIPTION OF SPECIFIC EMBODIMENTS

One or more specific embodiments of the present invention will be described below. In an effort to provide a concise description of these embodiments, not all features of an actual implementation are described in the specification. It should be appreciated that in the development of any such actual implementation, as in any engineering or design project, numerous implementation-specific decisions may be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Exemplary embodiments of the present invention are directed to automated control of medical devices to control at least one physiologic parameter (e.g., a blood oxygenation level, a tissue carbon dioxide level, a heart rate, a blood pressure level, a respiration rate, and/or a tissue oxygenation level) of a patient. While the exemplary embodiments of the present invention may control various different physiologic parameters, control of a patient's blood oxygen content is discussed below by way of example. One embodiment controls a composition and/or delivery amount of a gas mixture to a patient to safely induce, maintain, and/or control a patient's estimated blood oxygen saturation (i.e., SpO₂) level while taking into account circulatory delay. For example, automatic adjustment of FiO₂ by a computer-based controller may be utilized to control patient hypoxia or normoxia, and the controller may account for circulatory delays based on a sensor location (e.g., finger tip). It should be noted that FiO₂ may be defined as fractional inspired oxygen concentration or the percentage of oxygen in air inhaled by a patient through a ventilator. For example, in typical room air, the value for FiO₂ is approximately 21%.

A closed-loop FiO₂ controller in accordance with present embodiments may cooperate with a ventilator to control a patient's SpO₂ value. In a specific embodiment, the controller may receive input from a sensor (e.g., a pulse oximeter sensor) that measures the patient's SpO₂ value and, based on a comparison of the measured SpO₂ value with a target SpO₂ value, manipulate the ventilator's output. For example, the FiO₂ controller may increase FiO₂ when a measured SpO₂ value is below a predefined SpO₂ target or decrease FiO₂ when the measured SpO₂ value is above the SpO₂ target. By increasing or decreasing FiO₂, the patient's lungs receive more or less oxygen, respectively, and the value of SpO₂ in the patient will typically change correspondingly.

Human lungs exchange oxygen and carbon dioxide between the body and the environment. Specifically, the lungs oxygenate blood, which eventually carries the oxygen throughout the body. Accordingly, changes in FiO₂ may directly affect the oxygenation of blood in the lungs over several breaths and blood in other areas of the body shortly thereafter. Oximeter sensors generally do not measure the SpO₂ of blood near the lungs. For example, many non-invasive pulse oximeter sensors are adapted to photoelectrically sense blood constituents in the tissue of a patient's digits (e.g., fingers and toes), which are located a significant distance from the lungs. Because typical locations for SpO₂ measurement are a significant distance from the lungs, the blood in the tissue at typical measurement points may not immediately reflect changes induced by FiO₂ control in the overall blood oxygenation of the patient. Indeed, changes in blood oxygenation are generally not reflected in a measured SpO₂ value until newly oxygenated blood reaches the tissue at the measurement location from the lungs. Therefore, the SpO₂ level measured at the finger may suggest a low amount of oxygen in the patient's blood and cause an increased FiO₂ when, in fact, the SpO₂ of the lungs is at an acceptable level.

In addition to the location of SpO₂ measurement with respect to the lungs, it is now recognized that certain conditions may create additional physiologic delays. For example, peripheral vasoconstriction may result in circulatory delays of up to several minutes from when an aliquot of arterial blood is oxygenated in the lungs and when that same aliquot of arterial blood reaches an extremity (e.g., a fingertip), where the properties of the aliquot can be measured via pulse oximetry. Peripheral vasoconstriction may be defined as narrowing of the lumen in the blood vessels of a patient's extremities (e.g., fingers). Peripheral vasoconstriction may be induced by such common stimuli as prolonged exposure to cold air, certain vaso-active drugs, or the body's response to intravascular volume depletion. It should be noted that under the same vasoconstrictive stimuli, the circulatory delay between the lungs and a head site (e.g., forehead or ears) is substantially shorter than the delay between the lungs and extremities, such as a finger. Accordingly, embodiments of the present invention may take specific sensor location information into account.

In certain situations, prolonged physiologic delay (e.g., circulatory delay) can result in an unsteady physiologic parameter controller. For example, an FiO₂ controller may become unsteady when adjustments to FiO₂ are continually made based on SpO₂ measurements that are essentially inaccurate due to circulatory delay. Embodiments of the present invention compensate for control loop delays, including physiologic delays, to provide for a more stable control of certain patient physiologic parameters (e.g., SpO₂). For example, present embodiments include various implementations that mitigate potential controller instabilities by adjusting controller response time to compensate for physiologic delays, as will be discussed further below. By increasing control stability, embodiments of the present invention may prevent oscillations in patient physiologic parameters, allowing for improved and more efficient patient care.

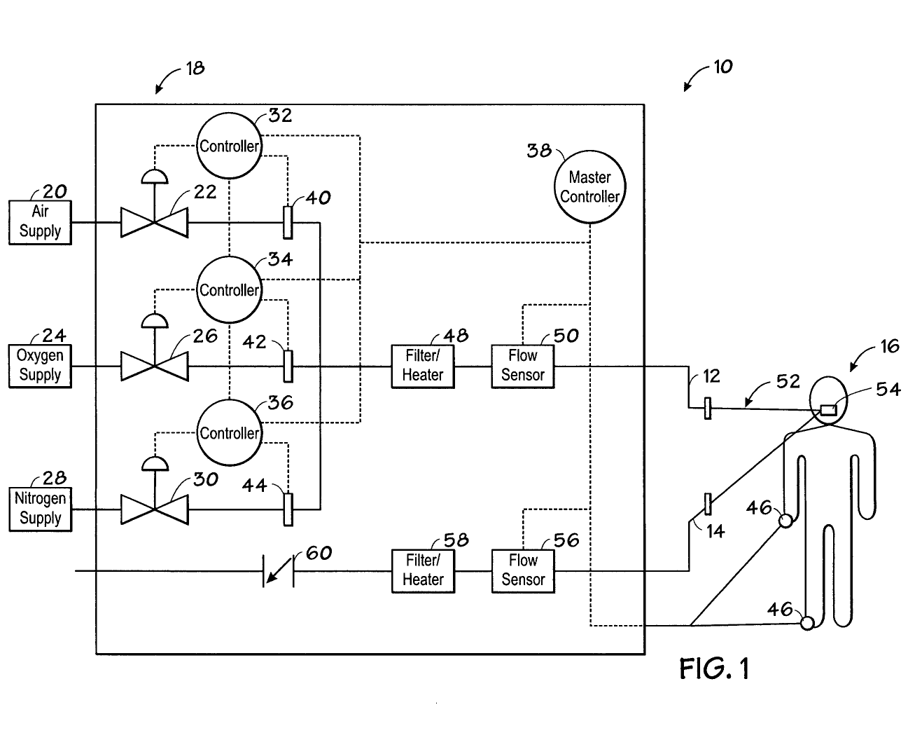

FIG. 1 is a block diagram of a ventilation system with a controllable gas mixture supply mechanism and a controller for maintaining or controlling a physiologic parameter while accounting for physiologic delay in accordance with embodiments of the present invention. The entire ventilation system is generally indicated by reference number 10. The ventilation system 10 may include a controller with a compensation scheme that corrects for control loop delays, including physiologic delays, to prevent instability in the controlled parameter.

The ventilation system 10 includes an inspiration line 12 and an expiration line 14. The inspiration line 12 provides a controlled gas mixture for a patient 16 to breathe. The expiration line 14 receives gases (e.g., oxygen and carbon dioxide) exhaled by the patient 16. It should be noted that in some embodiments the ventilation system 10 includes an open exhalation line rather than the expiration line 14. In embodiments that implement the open exhalation line, gases exhaled by the patient do not pass back through the ventilation system 10 but simply pass directly into the atmosphere. Depending on application requirements, the open exhalation line or the expiration line 14 may be utilized to provide for safe operation or to facilitate certain procedures.

An inlet portion 18 of the ventilation system 10 includes an air supply 20 coupled to an air valve 22, an oxygen supply 24 coupled to an oxygen valve 26, and a nitrogen supply 28 coupled to a nitrogen valve 30. The inlet portion 18 is designed to provide a defined gas mixture to the inspiration line 12. The supplies 20, 24, and 28 and valves 22, 26, and 30 may be utilized to produce normal, hyperoxic, and hypoxic gas mixtures for supply to the patient 16. Inclusion of the oxygen supply 24 may be desirable in some situations wherein a rapid increase in FiO₂ levels is desirable. However, it should be noted that some embodiments not requiring hyperoxic gas mixtures do not utilize the oxygen supply 24 but rely on the air supply for oxygen content in the gas mixture.

Each of the gas supplies 20, 24, and 28 may include a high pressure tank or cylinder with pressurized air, nitrogen, or oxygen disposed respectively therein. The valves 22, 26, and 30 and/or additional valves may operate to normalize the pressure and ensure desired gas mixture proportions. In one embodiment, the air supply 20 is the local atmosphere. That is, the air may be taken directly from the atmosphere using, for example, an air pump coupled to the air valve 22 in the inlet portion 18 of the ventilation system 10. Additionally, in some embodiments, a premixed gas supply is provided and regulated with a gas mixture valve that facilitates combination with air or oxygen. The premixed gas may be supplemented with oxygen, air or both, and it may eliminate the use of the nitrogen supply 28.

Each of the valves 22, 26, and 30 in the inlet portion 18 of the ventilation system may be a control valve, such as an electronic, pneumatic, or hydraulic control valve, that is communicatively coupled to a controller (e.g., flow controller or differential pressure controller), as illustrated by controllers 32, 34, and 36, respectively. The controllers 32, 34, and 36 may receive a set point value from a master controller 38 that controls SpO₂ levels in the patient 16. For example, each of the set points for the controllers 32, 34, and 36 may include a flow rate for each particular type of gas (e.g., air, oxygen, and nitrogen). To maintain or achieve a target SpO₂ level, the master controller 38 may supply set points or predefined curves (e.g., hysteresis curves) to the controllers 32, 34, and 36 such that levels of FiO₂ gradually fall or rise from a starting gas supply composition based on whether the patient needs more or less oxygen to reach the target SpO₂ level. The controllers 32, 34, and 36 may monitor flow sensors 40, 42, and 44 and open or close the valves 22, 26, and 30 depending on the amount of flow of each type of gas. These adjustments may maintain or control gas compositions in the inspiration line 12, as designated by the set points and/or curves from the master controller 38.

The illustrated controllers 32, 34, 36, and 38 may each include an input circuit configured to receive real-world data (e.g., a monitored physiological parameter of a patient) or other data (e.g., a set point from another controller). Additionally, the controllers 32, 34, 36, and 38 may each include an output circuit configured to provide signals (e.g., set point data) to a separate device or controller (e.g., controllers 32, 34, 36, and 38). For example, the output circuit may provide signals to an actuator or a set point value to a secondary controller (e.g., controller 32, 34, 36, and 38). Further, each controller 32, 34, 36, and 38 may include a memory storing an algorithm configured to calculate adjustments for maintaining or controlling physiologic parameters of the patient 16. Such algorithms (e.g., P, PD, PI, and PID algorithms) may be utilized to safely and efficiently bring the patient's physiological parameters to a desired state. In one exemplary embodiment, a control algorithm is implemented wherein a gas or gas mixture is delivered entirely from a single source at any given time. For example, based on a monitored physiological parameter, the control algorithm may alternate the single gas source after delivery of a defined volume, time period, or breath interval. Specifically, schemes such as those used in flow-conserving supplemental oxygen delivery devices or “oxygen conservers” may be utilized, thus simplifying the delivery mechanism and utilizing the patient's lungs to mix the gases from the various single sources.

The master controller 38 may be programmed to maintain or control SpO₂ levels in the patient 16 by providing the set points and/or curves to the controllers 32, 34, and 36 such that valves 22, 26, and 30 open or close to supply an appropriate gas mixture composition (e.g., increased FiO₂ to achieve a higher SpO₂). For example, the master controller 38 itself may have a steady or dynamic set point based on a physiological condition (e.g., blood saturation level) of the patient, as monitored by a sensor 46 or multiple sensors 46 that detect physiological conditions of the patient 16. For example, a set point of the master controller 38 may be a predefined estimated arterial oxygen saturation (SpO₂) level in the patient 16 or a continuously changing SpO₂ level. It should be noted that in some embodiments, a controller (e.g., master controller 38) may simply control the amount of gas supplied to the patient, rather than the composition, to control certain physiologic parameters.

In one embodiment, the master controller 38 may include a closed-loop FiO₂ controller configured to increase or decrease FiO₂ by an amount proportional to the difference between a measured SpO₂ value and an SpO₂ target value. Depending on the value of the proportion, which may be fixed or variable, the controller 38 may reach the SpO₂ target in a single step or in multiple steps. The master controller 38 may be designed to adjust the FiO₂ at an interval or controller cycle time that may be fixed or variable. Accordingly, the response time for the controller 38 to reach its SpO₂ target after a perturbation in FiO₂ may be determined by the controller cycle time and the number of cycles required to reach the target.

The master controller 38 may include a pulse oximeter used to derive SpO₂ levels, or alternatively, the master controller 38 may be coupled to a separate pulse oximeter (not shown). Accordingly, the sensor 46 or sensors 46 may include a pulse oximeter sensor and/or heart rate sensor that couples to the patient 16 to detect and facilitate calculation of the patient's SpO₂ and/or pulse. The sensor 46 may also include a temperature sensor to facilitate measurement of the patient's temperature at the sensor site. In one embodiment, the algorithm for determining the patient's SpO₂ is stored in a memory of the sensor 46. Similarly, algorithms relating to detection of physiologic delay may be stored in the memory of the sensor 46. Suitable sensors and pulse oximeters may include sensors and oximeters available from Nellcor Puritan Bennett Incorporated, as well as other sensor and pulse oximeter manufacturers.

A pulse oximeter and its associated sensors may be defined as a device that uses light to estimate oxygen saturation of pulsing arterial blood. For example, pulse oximeter sensors are typically placed on designated areas (e.g., a finger, toe, or ear) of the patient 16, a light is passed through designated areas of the patient 16 from an emitter of the pulse oximeter sensor, and the light is detected by a light detector of the pulse oximeter sensor. In a specific example, light from a light emitting diode (LED) on the pulse oximeter sensor may be emitted into the patient's finger under control of the pulse oximeter and the light may be detected with photodetector on the opposite side of the patient's finger. Using data gained through detecting and measuring the light with the pulse oximeter sensor, a percentage of oxygen in the patient's blood and/or the patient's pulse rate may be determined by the pulse oximeter. It should be noted that values for oxygen saturation and pulse rate are generally dependent on the patient's blood flow, although other factors may affect readings.

To control the patient's SpO₂ level, the master controller 38 may manipulate FiO₂ levels based on a comparison of one or more stored SpO₂ set points and/or curves with pulse oximetry measurements of the patient's SpO₂ level taken via the sensor 46. For example, if the patient's SpO₂ level is above a target level, the master controller 38 may reduce FiO₂ by increasing the amount of nitrogen feed (e.g., increasing flow through the nitrogen valve 30 by increasing the corresponding controller set point) while decreasing oxygen levels (e.g., decreasing flow through the oxygen and/or air valves 22 and 26 by decreasing the corresponding controller set points) in the inspiration line 12. Additionally, the master controller 38 may manipulate FiO₂ levels to control heart and respiration rates that are also being monitored by the sensors 46, which may include respiration sensors. For example, if the patient's heart rate exceeds a set value or if the respiration rate exceeds a set value, the master controller 38 may signal the gas supply controllers 32, 34, and 36 to increase FiO₂ by increasing oxygen related set points (e.g., flow rate of air) and decreasing non-oxygen gas related set points (e.g., flow rate of nitrogen).

The patient's measured value of SpO₂ may be significantly above or below the SpO₂ target value for various reasons. For example, the measured SpO₂ level may be below the target value when the controller 38 is first enabled, when a clinician increases the SpO₂ target value, or when the patient's SpO₂ drops due to hypopnea, apnea, or a large FiO₂ reduction. Low SpO₂ values may be especially frequent when a patient's ventilated lung volume is low. The measured value of the patient's SpO₂ may be significantly above the SpO₂ target value when the controller 38 is first enabled, when a clinician decreases the SpO₂ target value, when SpO₂ increases due to hyperventilation, or when SpO₂ increases due to a large FiO₂ increase.

In situations where the measured SpO₂ value is above or below the target value, the controller 38 will decrease or increase FiO₂, respectively, in an attempt to bring the SpO₂ to the target value. If there is a large difference in the measured SpO₂ value and target value, the increase or decrease in FiO₂ may be correspondingly large, which can cause large changes to the SpO₂ values. For example, if the measured SpO₂ value is well below the target SpO₂ value, the controller 38 may increase FiO₂ significantly to facilitate rapid attainment of the target value. If physiologic delays (e.g., circulatory delay) are not taken into account, such increases and decreases may be implemented for several control cycles before a corresponding increase or decrease in the SpO₂ value is detected. Indeed, as set forth above, it may take several minutes for blood in a patient's extremities to reflect the oxygen content of blood being oxygenated by the lungs. During this time, the patient may receive too little or too much oxygen based on the desired SpO₂ level.

If the response time of the controller is less than the response time of the sensor (including physiologic delay), the SpO₂ value may overshoot or undershoot the target SpO₂ value for several control cycles, as indicated above. Further, without adjustments for delay, over compensation may be perpetuated due to oscillations between oversupply and undersupply of FiO₂. For example, once the SpO₂ value at the measurement location catches up to the SpO₂ value near the lungs, the controller 38 may simply over adjust the FiO₂ again for several control cycles. Oscillations such as these may be disconcerting to a monitoring clinician and more importantly could have an impact on the efficiency and quality of patient care. Accordingly, embodiments of the present invention compensate controller response time to account for physiologic delays (e.g., circulatory delay).

To effectively mitigate the instability and oscillation discussed above, in accordance with embodiments of the present invention, the response time of the controller 38 is adjusted to exceed the circulatory delay between the lungs and the location of the SpO₂ sensor 46. This may be achieved by adjusting the control cycle time of the controller 38 or by adjusting the magnitude of the changes in FiO₂ made by the controller 38 based on the difference in the measured value of SpO₂ and the target value. For example, in one embodiment, a proportional component in the controller 38 may be set such that increases and decreases in FiO₂ are relatively small to prevent over adjustment during delays in response time including physiologic delays.

In one embodiment, adjustments to the controller response time are made based on an estimated amount of circulatory delay. For example, circulatory delay may be estimated by correlating changes in FiO₂ to subsequent changes in the measured SpO₂ value. These correlations may be made based on recent patient data stored while attempting to maintain the patient's SpO₂ level. However, in certain situations, estimating a value of the circulatory delay may require continuous or periodic changes in FiO₂ merely for the purpose of estimation. Theses changes may be undesirable because they may not facilitate maintenance of a certain level of patient oxygenation. Further, estimating a value of circulatory delay based on such information may also involve assuming that all SpO₂ changes are due to FiO₂ variations, which may be an inaccurate assumption. Indeed, SpO₂ changes may be the result of other physiologic changes. Accordingly, embodiments of the present invention may utilize other input to adjust response time.

Embodiments of the present invention may utilize an indication of sensor location to adjust the response time of the controller 38. Indeed, a user may enter the sensor location and/or the sensor 46 may be configured to facilitate determination of its location on the patient 16. For example, the sensor 46 may include a memory or a chip with a code indicating where the sensor 46 is designed to take measurements on the patient 16. In one embodiment, the sensor 46 may emit a signal that can be utilized to determine its precise location (e.g., coordinates). In another embodiment, the sensor 46 may indicate the sensor's model or type, which may be indicative of the recommended sensor site. In yet another embodiment, the sensor 46 may simply provide an indication of whether it is designed to take measurements near the patient's head or not. If the sensor 46 is located near the patient's head, the veins will not constrict as much, which may be taken into account when estimating potential circulatory delay. Indeed, if the sensor is determined to be away from the head, it will likely be on an extremity, and the controller 38 may use a slower response time to account for circulatory delay. It should be noted that in some embodiments, a user is able to confirm or deny an estimated location of the sensor 46 provided by the controller 38.

Embodiments of the present invention may also use a physiological indication (e.g., temperature and/or pulse amplitude) of local vasoconstriction to adjust the response time of the controller 38. Vasoconstriction may cause an area to cool because the blood is not flowing through the tissue to warm it. Accordingly, a temperature measurement below a designated value at the sensor location (e.g., a temperature of a patient's finger) may be an indicator of vasoconstriction. Further, low pulse amplitude may correspond to a small volume of blood flow, which may be indicative of vasoconstriction. Accordingly, a measurement of pulse amplitude below a designated limit may be an indicator of vasoconstriction.

These indications of vasoconstriction may be utilized in accordance with embodiments of the present invention to adjust the controller 38. For example, upon detecting a certain temperature or pulse amplitude, algorithms of the controller 38 may be adjusted to account for certain levels of circulatory delay. In one embodiment, upon detecting indications (e.g., low temperature or small pulse amplitudes) of circulatory delay, the controller 38 automatically slows its response time. In a specific example, a pulse oximeter may use a percentage of modulation in one or more wavelengths of light due to the patient's pulse amplitude along with data averaging and pulse qualification algorithms to asses a degree of local vasoconstriction and adjust algorithm parameters accordingly. It should be noted that the indications of local vasoconstriction and the sensor site may be utilized in conjunction or separately to make controller adjustments in accordance with embodiments of the present invention. Furthermore, circulatory delay might be estimated at two or more SpO₂ sensor sites, and SpO₂ values corresponding to the lowest delay to control FiO₂ adjustments.

After being mixed or flowed according to the set points determined by master controller 38, the gas mixture proceeds from the inlet portion 18 of the ventilation system 10 along the inspiration line 12 to a filter/heater 48. The filter/heater 48 may operate to filter out bacteria, remove other potentially harmful or undesirable elements, and heat the gas mixture to a desired temperature. Upon exiting the filter/heater 48, the gas mixture may proceed to a flow sensor 50 (e.g., a differential pressure sensor) that measures a total flow rate of the gas mixture to the patient 16 through the inspiration line 12. Values obtained from the flow sensor 50 may be utilized in control and maintenance of patient SpO₂ levels by providing information for use in algorithms of the master controller 38 and/or other controllers 32, 34, and 36. Eventually, the gas mixture exits the ventilation system 10 via tubing 52 for delivery to the patient 16 via a delivery piece 54 (e.g., endotracheal tube, laryngeal mask airway, face mask, nasal pillow, or nasal canula).

Several implementations of the expiration line 14 may be utilized to handle gases (e.g., CO₂ and O₂) exhaled by the patient 16. For example, different exhalation sensors, filters, heaters, and configurations may be utilized dependent upon the patient's needs and/or other desirable conditions. In the embodiment illustrate by FIG. 1, gases exhaled by the patient 16 are received back into the ventilation system 10 via the expiration line 14. Once received, the exhaled gases proceed through a flow sensor 56, which measures values associated with the exhaled gases (e.g., a volumetric flow rate). Information from the flow sensor 56 may be utilized to further adjust parameters that relate to safely maintaining patient SpO₂ levels. For example, flow rates of exhaled air from the patient may be utilized in an algorithm of the master controller 38 to compare with a predefined minimum exhalation rate for the patient. A difference between flow sensors 50 and 56 may be used to determine when the patient inspires or exhales, the patient's inspired or exhaled flow may then be integrated to determine inspired or exhaled volume. Respiratory parameters such as respiratory rate and minute volume may then be calculated and used as auxiliary inputs to the master controller 38.

Upon exiting the flow sensor 56, the exhaled gas may proceed to a filter/heater 58, to a check valve 60, and out of the ventilation system 10. The filter heater may be adapted to cleanse the exhaled gases, and the check valve 60 may operate to prevent the exhaled gases from circulating back to the patient 16 through the ventilation system 10.

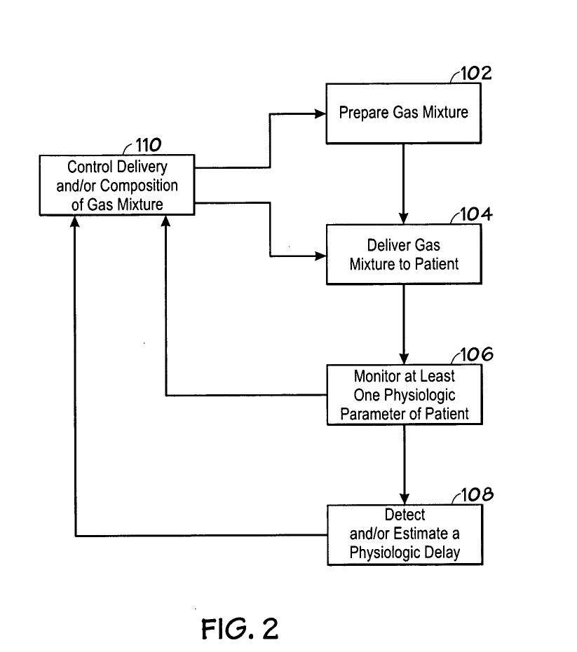

FIG. 2 is a block diagram of a method illustrating an exemplary embodiment of the present invention. The method is generally referred to by reference number 100. Specifically, method 100 begins with preparation of a breathable gas mixture (block 102). For example, block 102 may include mixing gases from the supplies 20, 24, and 28 in the inlet portion 18 of the ventilation system 10 to maintain a certain FiO₂ using the controllers 32, 34, 36, and 38, and valves 22, 26, and 30 based on data received from the sensors 46, 50, and 56. In other embodiments, block 102 may include manipulating an amount of gas (e.g., reducing or increasing flow) provided to a patient to maintain a certain FiO₂. Next, block 104 represents delivering the gas mixture to a patient, as may be achieved via the inspiration line 12 of the ventilation system 10 illustrated by FIG. 1.

Block 106 represents monitoring at least one parameter (e.g., SpO₂) of the patient. Block 108 represents detecting and/or estimating a physiologic delay (e.g., circulatory delay) based on the at least one physiological parameter. For example, an estimation of physiologic delay may be based on historical changes in SpO₂. In one embodiment, multiple parameters are monitored and utilized to estimate the physiologic delay. For example, in one embodiment, temperature and/or pulse amplitude may be utilized to estimate a difference in time required for FiO₂ changes to register in blood near a patient's lungs and at a sensor location on the patient. It should be noted that block 108 may represent an actual estimated degree of delay or a simple determination that such a delay is likely present. For example, block 108 may represent determining that a certain degree of local vasoconstriction is present and causing delay and/or an amount (e.g., minutes) of delay caused by the vasoconstriction.

Block 110 represents controlling the delivery of the gas mixture to the patient based on the at least one physiological parameter and the estimation or detection of physiologic delay. For example, this can be achieved using the master controller 38 of the ventilation system 10. By continually monitoring patient physiological parameters and updating input variables, as illustrated by block 110, embodiments of the present invention may maintain or control certain physiologic parameters, such as a patient's SpO₂ level. In some embodiments, other procedures are also implemented to facilitate, improve, or achieve diagnostic and/or therapeutic results.

While the invention may be susceptible to various modifications and alternative forms, specific embodiments have been shown by way of example in the drawings and will be described in detail herein. However, it should be understood that the invention is not intended to be limited to the particular forms disclosed. Rather, the invention is to cover all modifications, equivalents and alternatives falling within the spirit and scope of the invention as defined by the following appended claims.

1. A method for automatically controlling blood oxygen saturation in a patient, comprising:
delivering a gas mixture to the patient;
monitoring at least one physiologic parameter of the patient;
determining a physiologic delay between delivering the gas mixture and detection of a corresponding response to delivering the gas mixture; and
automatically controlling the delivery of the gas mixture based at least in part upon the delay detected and based on a value of the physiologic parameter.

2. The method of claim 1, wherein the gas mixture is delivered at least in part via an endotracheal tube, laryngeal mask airway, face mask, nasal pillow, nasal canula, or any combination thereof.

3. The method of claim 1, wherein the at least one physiological parameter comprises a blood oxygenation level, a tissue carbon dioxide level, a heart rate, a blood pressure level, a respiration rate, a tissue oxygenation level, or any combination thereof.

4. The method of claim 1, wherein determining the physiologic delay comprises estimating an amount of the physiologic delay.

5. The method of claim 1, comprising receiving an indication of physiologic delay from a sensor.

6. The method of claim 1, wherein determining the physiologic delay comprises receiving a sensor identification code, a temperature, a pulse amplitude, or location coordinates.

7. The method of claim 1, wherein determining the physiologic delay comprises detecting oscillations in the at least one physiologic parameter.

8-23. (canceled)

24. A gas supply device for automatically controlling blood oxygen saturation in a patient, comprising:
means for delivering a gas mixture to the patient;
means for monitoring at least one physiological parameter of the patient;
means for determining a physiological delay between delivering the gas mixture and detection of a corresponding response to delivering the gas mixture; and
means for automatically controlling the delivery of the gas mixture based at least in part upon the delay detected and based on a value of the physiological parameter.

25. The gas supply device of claim 24, wherein the means for delivering the gas mixture comprises an endotracheal tube, laryngeal mask airway, face mask, nasal pillow, nasal canula, or any combination thereof.

26. The gas supply device of claim 24, wherein the at least one physiological parameter comprises a blood oxygenation level, a tissue carbon dioxide level, a heart rate, a blood pressure level, a respiration rate, a tissue oxygenation level, or any combination thereof.

27. The gas supply device of claim 24, wherein the means for determining the physiological delay comprises means for establishing an amount of the physiological delay.

28. The gas supply device of claim 24, comprising means for receiving an indication of physiological delay from a sensor.

29. The gas supply device of claim 24, wherein the means for determining the physiological delay comprises means for receiving a sensor identification code, a temperature, a pulse amplitude, or location coordinates.

30. The gas supply device of claim 24, wherein the means for determining the physiological delay comprises means for detecting oscillations in the at least one physiological parameter.
